(19) United States
(12) Patent Application Publication
(10) Pub. No.: US 2022/0084673 A1
(43) Pub. Date: Mar. 17, 2022

(54) INFORMATION PROCESSING APPARATUS, INFORMATION PROCESSING METHOD AND INFORMATION PROCESSING PROGRAM

(71) Applicant: CureApp, Inc., Tokyo (JP)

(72) Inventors: Shin Suzuki, Tokyo (JP); Ryuhei So, Tokyo (JP)

(21) Appl. No.: 17/499,943

(22) Filed: Oct. 13, 2021

Related U.S. Application Data
(63) Continuation of application No. PCT/JP2021/025760, filed on Jul. 8, 2021.

(30) Foreign Application Priority Data
Sep. 16, 2020 (JP) 2020-155870

(51) Int. Cl. G16H 40/67; H04L 12/58

(52) U.S. Cl. CPC G16H 40/67; H04L 51/02

(57) ABSTRACT

An information processing apparatus, an information processing method, and an information processing program capable of providing contents in accordance with preferences of a user. The information processing apparatus includes a reception unit that receives input information based on input related to medical treatment in a patient terminal; a selection unit that selects at least one chatbot in accordance with a patient from among a plurality of chatbots that differ in responses related to the medical treatment for the patient; an acquisition unit that acquires response information for making a response related to the medical treatment to the input information received by the reception unit in accordance with the characteristics of the chatbot selected by the selection unit; and a communication unit that transmits the response information acquired by the acquisition unit to the patient terminal.

### TECHNICAL FIELD

This disclosure relates to an information processing apparatus, an information processing method, and an information processing program.

### BACKGROUND

Systems that assist users in managing their health have been used conventionally. The system includes terminals and a server. Each terminal obtains the biometric information of the user and behavioral information about the behavior of the user and transmits them to the server. Japanese Laid-Open Patent Publication No. 2019-168957 discloses a server selects and transmits to a terminal an evaluation message that evaluates health management efforts of a user for each of management items based on biometric and behavioral information of the user and evaluation criteria for the management items related to the health management of the user.

However, JP2019-168957A only transmits the evaluation message selected based on the biometric and behavioral information from the server to the terminal, and does not transmit an evaluation message that matches the characteristics of the user. That is, each user has his or her own preferences, and the server may transmit an evaluation message to the terminal that the user does not prefer. Thus, the user may refrain from accepting the contents of the evaluation message.

It could therefore be helpful to provide an information processing apparatus, an information processing method, and an information processing program capable of providing contents in various forms.

### SUMMARY

We thus provide:

An information processing apparatus includes: a reception unit that receives input information based on input related to medical treatment in a patient terminal; a selection unit that selects at least one chatbot in accordance with a patient from among a plurality of chatbots having different responses related to the medical treatment for the patient; an acquisition unit that acquires response information related to the medical treatment for responding to the input information received by the reception unit in accordance with the characteristics of the chatbot selected by the selection unit; and a communication unit that transmits the response information acquired by the acquisition unit to the patient terminal.

The selection unit may select at least one chatbot from among a plurality of chatbots having different characters in at least one of appearance, manner of response, and voice of response.

The selection unit may select one of a plurality of chatbots having different time points of transmitting the response information to the patient terminal and different manners of communication to the patient recorded in the response information as well as different settings as the characteristics of the chatbot in accordance with the preferences of the patient that owns the patient terminal.

The reception unit may receive input information on medical treatment for the patient that owns the patient terminal, and the acquisition unit may acquire response information as a proposal to improve a disease as a response regarding the medical treatment for the patient.

The reception unit may receive the behavioral input information on the behavior of the patient, and the selection unit may change the chatbot to be selected based on the response information and the behavioral input information.

The reception unit may receive, as the behavioral input information, behavior performed by the patient in response to the response information transmitted to the patient terminal by the communication unit.

The selection unit may change the character to be selected based on the learning model that has learned in advance the response information in accordance with the characteristics of the chatbot and the behavior performed by the patient in response to the response information, and the behavioral input information received by the reception unit.

The reception unit may receive the behavioral input information from an external device in which the behavioral input information on the behavior of the patient has been stored.

An information processing method causes a computer to perform the steps of: receiving input information based on input related to medical treatment in a patient terminal; selecting at least one chatbot in accordance with a patient from among a plurality of chatbots having different responses related to the medical treatment for the patient; acquiring response information on the medical treatment for responding to the input information received in the receiving step in accordance with the characteristics of the chatbot selected in the selecting step; and transmitting the response information acquired in the acquiring step to the patient terminal.

An information processing program causes a computer to execute the functions of: receiving input information based on input related to medical treatment in a patient terminal; selecting at least one chatbot in accordance with a patient from among a plurality of chatbots having different responses related to the medical treatment for the patient; acquiring response information on the medical treatment for responding to the input information received by the reception function in accordance with the characteristics of the chatbot selected by the selection function; and transmitting the response information acquired by the acquisition function to the patient terminal.

An information processing apparatus receives input information on a medical treatment for a patient, selects at least one chatbot in accordance with the patient from among a plurality of chatbots having different responses related to the medical treatment for the patient, and acquires and transmits to a patient terminal response information on the medical treatment in response to the input information in accordance with the characteristics of the selected chatbot, thereby providing contents in a form in accordance with the preferences of the user.

The information processing method and the information processing program may have the same advantages as those of the information processing apparatus described above.

### BRIEF DESCRIPTION OF THE DRAWINGS

Features, advantages, and technical and industrial significance of representative examples will be described below with reference to the accompanying drawings, in which like numerals denote like elements.

FIG. 1 is a diagram illustrating an information processing system according to an example.

FIG. 2 is a block diagram illustrating an information processing apparatus according to an example.

FIG. 3 is a diagram illustrating an example of an image displayed on a display of a patient terminal.

FIG. 4 is a flowchart illustrating an information processing method according to an example.

### DETAILED DESCRIPTION

A selected, representative example will be described below.

The word “information” is used, but the word “information” may be rephrased as “data” and the word “data” may be rephrased as “information.”

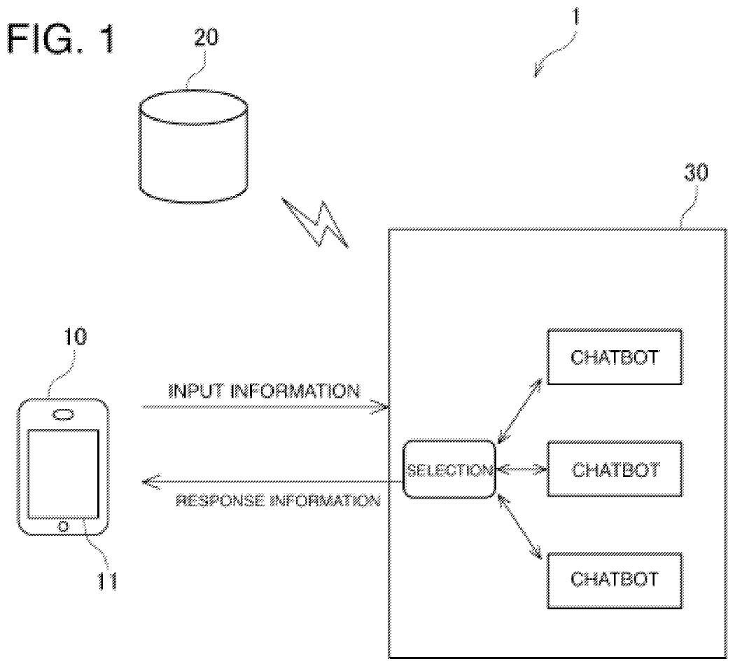

FIG. 1 is a diagram illustrating an information processing system 1 according to an example.

The information processing system 1 performs chatting between terminals and an information processing apparatus 30. The information processing system 1 provides medical information from the information processing apparatus 30 to a patient (user) using the terminal through the chatting. As an example, when the information processing system 1 advises a patient on lifestyle diseases, the system 1 transmits to the terminal a response (response information) to improve the lifestyle depending on the information input by the patient (input information).

Such an information processing system 1 includes the terminals and the information processing apparatus 30.

The terminals each are directed to, for example, a patient terminal 10 used by the patient. The “terminal” is hereinafter referred to as “patient terminal 10.” Specifically, the patient terminal 10 may be a smartphone, a tablet, a laptop, and a personal computer, for example. Texts and symbols may be input into the patient terminal 10 on the basis of the operation of the patient. The patient terminal 10 transmits the input information to the information processing apparatus 30.

The information processing apparatus 30 communicates, for example, with the patient terminal 10. The information processing apparatus 30 may be directed to, for example, a server, a personal computer, a laptop, a tablet, and a smartphone. The information processing apparatus 30 includes a plurality of chatbots. The information processing apparatus 30 responds to the input information transmitted from the patient terminal 10 by using the chatbot. That is, the information processing apparatus 30 transmits the response information to the patient terminal 10. In this configuration, the information processing apparatus 30, as an example, selects one of the plurality of chatbots in accordance with the behavior of the patient and responds to the patient in accordance with the characteristics of the selected chatbot. The information processing apparatus 30 may change the chatbot in the middle of the chatting.

Each of the plurality of chatbots described above may have a different character, e.g., a doctor, a nurse, a friend, a family member or the like. Depending on the character, the chatbot responds to the input of the patient in different ways and at different time points. As an example, if the chatbot is a doctor character, it responds to the patient as if it were a doctor examining the patient. In addition, as an example, if the chatbot is a friend, it responds to the patient in the same way that a friend would respond to a friendly conversation. When the chatbot is a friend character, the chatbot may be set as, for example, a companion that is treated with the patient (as an example, a senior that has overcome the disease of the patient, and a companion undergoing treatment). The information processing apparatus 30 selects the chatbot when the patient will accept and act on the contents of the chatbot.

The information processing apparatus 30 will be described in detail below.

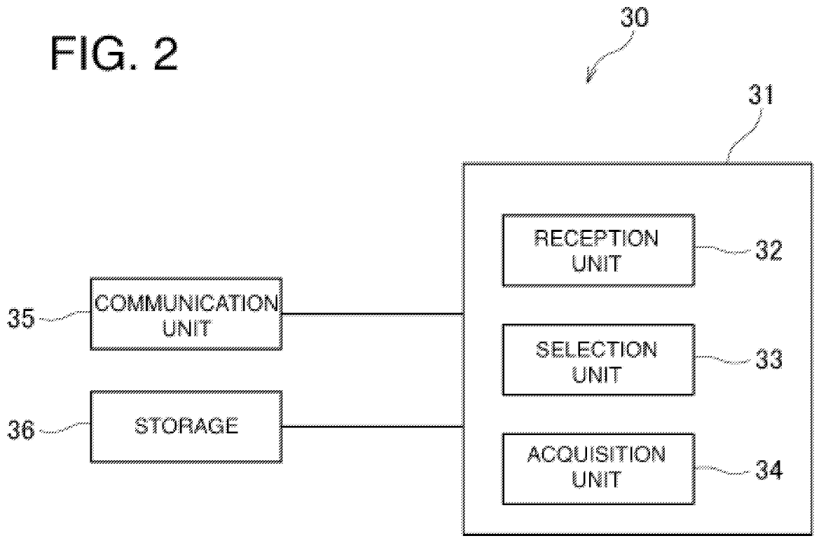

FIG. 2 is a block diagram illustrating the information processing apparatus 30 according to an example.

The information processing apparatus 30 includes a communication unit 35, a storage 36, a reception unit 32, a selection unit 33, and an acquisition unit 34. The reception unit 32, the selection unit 33, and the acquisition unit 34 may be embodied as one function of the controller 31 (as an example, an arithmetic processing unit) of the information processing apparatus 30.

The communication unit 35 communicates with the patient terminal 10. The communication unit 35 may also communicate with an external device other than the patient terminal 10 (such as the server 20 (see FIG. 1) as an example). The communication unit 35 receives input information from the patient terminal 10 as described below. The communication unit 35 receives behavioral input information from the patient terminal 10 or the server 20 as described below. The communication unit 35 transmits response information acquired by the acquisition unit 34 to the patient terminal 10 as described below.

The storage 36 stores various information and programs. For example, the storage 36 stores information and programs related to the chatbots.

The reception unit 32 receives input information based on inputs related to medical treatment in the patient terminal 10. In this configuration, the reception unit 32 may receive input information on medical treatment for the patient that owns the patient terminal 10. The input (input information) related to medical treatment may include contents related to the patient's own medical treatment, and as one specific example, may be various contents including alcoholism, insomnia, obesity, and headaches. The input information may correspond to the results of examinations related to the medical treatment on the patient.

The reception unit 32 may receive behavioral input information on the behavior of the patient. That is, the reception unit 32 may receive, as the behavioral input information, behavior performed by the patient in response to the response information transmitted to the patient terminal 10 by the communication unit 35. In this example, the reception unit 32 may receive the behavioral input information from an external device that has stored the behavioral input information on the behavior of the patient.

When the chatting is performed between the information processing apparatus 30 (chatbot) and the patient terminal 10, the reception unit 32 receives the contents of the behavior (behavioral input information) of the patient when the patient behaves in response to the contents of the response of the information processing apparatus 30 (chatbot). That is, the reception unit 32 receives, for example, behavioral input information regarding whether the patient has behaved in accordance with the contents of the response of the information processing apparatus 30 or whether the patient has not followed the contents of the response.

When the patient terminal 10 transmits the behavioral input information to the server 20, the reception unit 32 may receive the behavioral input information transmitted from the server 20.

For example, the reception unit 32 may receive the behavioral input information after undergoing the following processes.

That is, as an example, if the response information to be described below, which is transmitted from the information processing apparatus 30 to the patient terminal 10, is directed to “encouraging exercise,” and the exercise taken by the patient in response is a walk, the patient terminal 10 may measure the number of steps taken during the walk. The patient terminal 10 may associate the information of the measured number of steps (behavioral input information) with the identification information that identifies the patient and transmit the information to the server 20 (or the information processing apparatus 30). The server 20 may store the behavioral input information and the identification information when received and transmit the behavioral input information and the identification information to the information processing apparatus 30 at a predetermined time point.

The selection unit 33 selects at least one chatbot from among a plurality of chatbots with different responses related to the medical treatment to the patient in accordance with the patient. The selection unit 33 may select at least one chatbot from among a plurality of chatbots having different characters in at least one of appearance, manner of response, and voice of response. A plurality of chatbots has different characteristics as described above. As an example, each of the plurality of chatbots may have a different character, e.g., a doctor, a nurse, a friend, a family member or the like. Depending on the character, the chatbot may respond to the input of the patient in different ways and at different time points, for example. Each of the plurality of chatbots responds to the same contents by different expressions. When the information processing apparatus 30 (chatbot) transmits the response information to the patient terminal 10, the display 11 of the patient terminal 10 displays a character. For example, if the chatbot is a doctor, the display 11 of the patient terminal 10 displays a picture (appearance) of the doctor.

The appearance may be, for example, a picture of the chatbot that is displayed on the display 11 of the patient terminal 10.

The response may be in various ways, including, for example, a response such as examining a patient if the chatbot is a doctor character.

The time point of the response may be various, for example, when the reception unit 32 receives the input information, the response information may be immediately transmitted. Alternatively, the time points from the reception of the input information by the reception unit 32 to the transmission of the response information may vary from time to time.

The voice of the response may be the voice output from the patient terminal 10 when the response information of the information processing apparatus 30 (chatbot) is voice output by the patient terminal 10. The voice of the response may be different, for example, male voice, female voice, low voice and high voice.

The selection unit 33 may select one of a plurality of chatbots that differ in the characteristics of the chatbots such as the time points of transmitting the response information to the patient terminal 10 and the method of communicating to the patient recorded in the response information, as well as the settings of the chatbots in accordance with the preferences of the patient that owns the patient terminal 10. The preferences show what type of chatbot the patient prefers. The preferences of the patient may include, for example, indicators of the personality (e.g., neurotic, and impulsive, as examples) of the patient and the senses of values (e.g., physical health-minded, valuing human relations, and economy, as examples) of the patient.

For example, when the selection unit 33 selects a chatbot based on the operation on the patient terminal 10 by the patient in accordance with the preferences of the patient, the selection unit 33 may select a chatbot in accordance with the selection.

Alternatively, the selection unit 33 may select a chatbot in accordance with a response of the patient to a questionnaire (question) displayed on the display 11 of the patient terminal 10. The questionnaire (questions) may, for example, be designed to investigate the preferences of the patient.

The selection unit 33 may, for example, select any chatbot from among a plurality of chatbots when the input information is received from the patient terminal 10 for the first time. When the input information is received from the patient terminal 10 for the second or later time, for example, the selection unit 33 may change the chatbot to be selected based on the response information and the behavioral input information. Alternatively, the selection unit 33 may, for example, use the chatbot selected for the first time until the preferences of the patient are acquired based on the behavioral input information and the input information for the response information, and change the chatbot in accordance with the preferences of the patient when the preferences of the patient are acquired. The behavioral input information is received by the reception unit 32 as described above. When the chatting is performed between the information processing apparatus 30 (chatbot) and the patient terminal 10, the selection unit 33 changes the chatbot based on the contents of the response of the information processing apparatus 30 (chatbot) and the contents of the behavior (behavioral input information) of the patient when the patient behaves in response to the contents of the response. For example, when the response information is transmitted from the information processing apparatus 30 to the patient terminal 10 and the behavioral input information is not received by the reception unit 32 even after a predetermined time has elapsed, the selection unit 33 may presume that the chat application has not been run on the patient terminal 10 and change the chatbot. In addition, the selection unit 33 may change the chatbot when, after selecting one chatbot, the symptoms of the patient estimated based on the input information and the behavioral input information do not improve even after a predetermined period of time has elapsed.

The selection unit 33 is allowed to change the chatbot at various time points, i.e., at any time.

The selection unit 33 may change the character to be selected based on the learning model that has learned in advance the response information in accordance with the characteristics of the chatbot and the behavior performed by the patient in response to the response information, and the behavioral input information received by the reception unit 32. That is, the controller 31 generates a learning model by learning in advance the chatbots, the contents of the response information transmitted to the patient terminal 10 based on each of the plurality of chatbots, and the behavior performed by the patient in response to the response information. When the behavioral input information is received by the reception unit 32, the selection unit 33 changes the chatbot to a chatbot suitable for the behavior of the patient based on the response information, the behavioral input information, and the learning model.

The acquisition unit 34 acquires the response information on the medical treatment for responding to the input information received by the reception unit 32 in accordance with the characteristics of the chatbot selected by the selection unit 33. The response information is stored in the storage 36 in advance. The contents of the input are associated with the contents of the response in advance, allowing the acquisition unit 34 to acquire the response information corresponding to the input information from the storage 36.

In this configuration, the controller 31 may generate a learning model by learning the contents of the input and the contents of the response in advance. The acquisition unit 34 acquires the response information from the storage 36 based on the input information and the learning model. The response information is directed to, for example, information that is a suggestion to improve the symptoms of the patient and indicates a response regarding the medical treatment for the patient.

As one specific example, when improving the alcohol dependence of the patient, the response information may be associated with the image of alcohol and the phrase “I hate alcohol.” The input (input) of the patient to the response information allows the information processing apparatus 30 to presume whether the patient actually wishes to stop drinking.

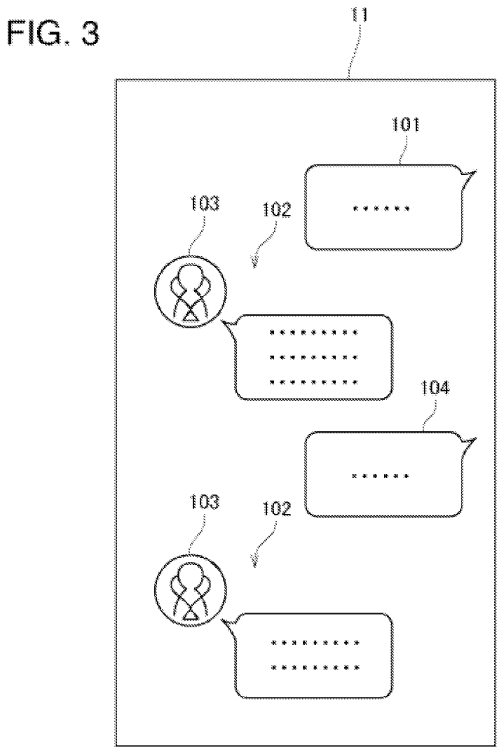

FIG. 3 is a diagram illustrating an example of an image displayed on the display 11 of the patient terminal 10.

Symptoms, for example, may be entered into the patient terminal 10 by the patient operating an input unit (not shown). For example, when improving the symptoms of the lifestyle disease of the patient, the lifestyle and the current symptoms of the patient are entered into the patient terminal 10. The patient terminal 10 displays the entered information 101 (input information) such as texts and symbols on the display 11. The patient terminal 10 also transmits the input information to the information processing apparatus 30.

The information processing apparatus 30 responds to the input information. That is, the information processing apparatus 30 transmits the response information to the patient terminal 10. In this response, the information processing apparatus 30 transmits the response information in accordance with the characteristics of the chatbot selected by the selection unit 33. For example, when the character of the chatbot is a doctor, the information processing apparatus 30 transmits the response information to the patient terminal 10 in a tone and at a time point such that the doctor talks to the patient, as one specific example.

The patient terminal 10 displays texts and images 102, for example, based on the response information on the display 11. In this configuration, the patient terminal 10 may display an image (icon 103) corresponding to the character of the chatbot on the display 11. For example, if the character of the chatbot is a doctor, an image (icon 103) of the doctor may be displayed on the display 11 adjacent to the response information (text). Regarding the expression of the icon 103, as an example, if the icon is an image of a doctor, the expression of the doctor may change. In addition, regarding the appearance of the icon, as an example, if the icon is a companion that is being treated together, the appearance of the companion may change.

When the response of the patient to the response information is input based on the operation of the patient, the patient terminal 10 displays the response 104 on the display 11. The response of the patient may include, as an example, a response to a question when the response information is directed to a question from the information processing apparatus 30, for example. In addition, when the response information prompts the patient to perform the behavior (e.g., exercise and medication to improve the lifestyle), the response of the patient corresponds to the behavior performed by the patient in response (e.g., “went jogging” and “took medication”). The patient terminal 10 transmits the responses of the patient as the input information and behavioral input information to the information processing apparatus 30.

The information processing apparatus 30 transmits the response information to the response 104 (input information) transmitted from the patient terminal 10 in the same manner as described above.

For example, when the information processing apparatus 30 transmits the response information to the patient terminal 10 and receives the input information as a response from the patient terminal 10, the information processing apparatus 30 records the behavioral input information for the behavior that the patient responds with the input information. That is, the information processing apparatus 30 records the behavioral input information in that “The patient is willing to improve the symptoms.” In addition, when the information processing apparatus 30 receives “behavior performed by a patient in response to a prompt for behavior to the patient” from the patient terminal 10, the information processing apparatus 30 records information on the behavior of the patient (i.e., behavioral input information). The information processing apparatus 30 may, for example, refrain from changing the character of the chatbot if the behavioral input information is recorded. In contrast, the information processing apparatus 30 may, for example, change the character of the chatbot if the behavioral input information is not recorded. This change may be, for example, directed to changing the character of the chatbot from “a doctor” to “a nurse.”

Next, a specific example of the information processing system 1 will be described.

In the following, example in which the patient continues abstinence from drinking for a long time and in which the patient reduces alcohol consumption will be described.

First, the medical professional (instructor) and the patient have a discussion at the time when the patient enters a hospital by asking about the sense of values that motivates the patient to continue abstinence from drinking and reduce alcohol consumption and asking about the advantages and disadvantages of drinking.

The health care provider then suggests location- and time-specific measures to the patient or makes new suggestions if the patient fails to implement the measures.

The information processing system 1 has a function of allowing the patient to consult with at the time point when the desire of the patient for alcohol increases between this attendance and the next attendance. For example, the information processing system 1 is capable of providing a social pressure role-play in which a patient drinks in response to an invitation to a drinking party from a friend. In this example, the information processing system 1 may display a plurality of options on the patient terminal 10 and transmit response information from the information processing apparatus 30 (chatbot) in accordance with the option selected based on the operation of the patient. The options and the contents of the response are associated with each other in advance.

The chatbots may include several types of chatbots with different characteristics. When one of a plurality of chatbots is selected, a phase in which the sense of values and preferences (tastes) of the patient are ascertained may be prepared first, and the sense of values (preferences) of the patient may be presumed through the dialogue between the patient and the chatbot in that phase. That is, the chatbot conducts a motivational interview with the patient. As one specific example, when a chatbot asks the patient “What prompted you to take this program?” and the patient responds “I was having trouble getting work done,” the chatbot may respond, “I see that you were having trouble getting work done.” The chatbot asks questions that cause the patient to repeat the responses to them, allowing the chatbot to understand the sense of values and preferences of the patient. In the phase, the information processing system 1 may select a chatbot that suits the patient by allowing the patient to interact with a plurality of chatbots with different characteristics. The chatbot may also be selected using a learning model that has learned the characteristics of the patients and the chatbots that fit the patients with those characteristics in advance.

As described above, the information processing system 1 is capable of providing characters such as a doctor, a nurse, a friend and a family member as chatbots. When, for example, a doctor is selected as a chatbot based on the motivational interview described above, the information processing system 1 may select a chatbot with a communication style that gives strong verbal instructions to the patient if the symptoms of the patient fail to improve despite the establishment of a relationship of mutual trust between the chatbot and the patient.

In contrast, when a companion is selected as the chatbot, for example, the information processing system 1 may provide a character of “a senior that had the same symptoms as those of the patient but improved them” as the chatbot. In addition, the information processing system 1 may, for example, provide a character of “a junior that has the same symptoms as those of the patient but fails to improve the symptoms” as a companion. The information processing system 1 may provide a chat in which a senior and a junior as characters have a conversation, for example, and when the junior remarks that he cannot improve his symptoms, the senior remarks about his experience in improving his symptoms. The information processing system 1 may encourage the patient to take action to improve his or her symptoms by providing the patient with such a chat between a senior and a junior as companions to improve the symptoms together with the patient.

Even if a doctor is selected as the character, the information processing system 1 may change the character in the chat based on the statements of the patient in the chat. The information processing system 1 may, for example, have an internist appear if the patient has a symptom related to the body, or a psychiatrist if the patient has a symptom related to the mind.

When coaching is performed on the patient from the information processing apparatus 30, the information processing system 1 may repeatedly transmit the same contents (response information) to the patient terminal 10 to fix them in the memory of the patient. In this example, when the information processing system 1 transmits the same contents (response information) to the patient terminal 10, the information processing system 1 may transmit the response information with different wordings to the patient terminal 10.

The information processing system 1 may change the contents (response information) to be transmitted to the patient terminal 10 in accordance with the phase of the interaction between the patient and the chatbot and the proficiency of the patient. In addition, the information processing system 1 may ask the same question repeatedly to allow the patient to declare the correct contents.

The information processing system 1 may register the contents of the declaration (input information) of the patient acquired by the information processing apparatus 30 in an electronic medical record used by a medical professional. In the information processing system 1, the chatbot may be operated by a medical professional. For example, the information processing apparatus 30 may transmit the contents entered to the information processing apparatus 30 by a medical professional to the patient terminal 10 via a chatbot. The icon 103 displayed on the display 11 of the patient terminal 10 may be an image that resembles a medical professional in charge of the patient such as the general practitioner of the patient.

The chatbot may interact with multiple patients at the same time. In this example, the chatbot may be operated by a medical professional. That is, the information processing system 1 may allow the medical professional and the patient to interact with each other via a chatbot.

Next, an information processing method according to an example will be described.

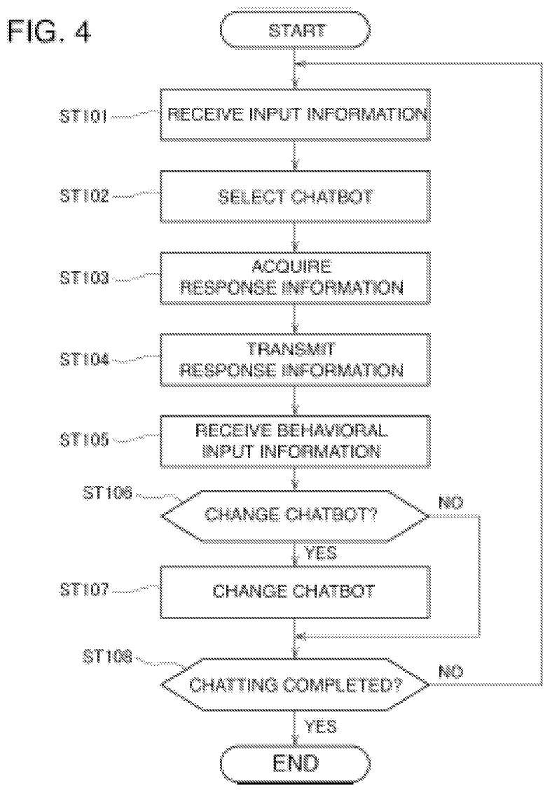

FIG. 4 is a flowchart illustrating an information processing method according to an example.

In step ST101, the reception unit 32 receives input information in the patient terminal 10. The input information is generated in the patient terminal 10 and transmitted to the information processing apparatus 30. The input information may be contents related to various medical treatment (cure) including symptoms, for example, of the patient that owns the patient terminal 10.

In step ST102, the selection unit 33 selects the chatbot. In this example, the selection unit 33 may select a chatbot in accordance with the preferences of the patient based on, for example, a questionnaire for the patient and the results of examination of the patient performed by the medical professional. The selection unit 33 may, as an example, select the chatbot using a learning model that has learned the preferences of the patient, the types of chatbots, and the treatment continuation rate and treatment effect of the patient in advance.

In step ST103, the acquisition unit 34 acquires the response information corresponding to the input information received in step ST101. The acquisition unit 34 may, for example, acquire the response information corresponding to the input information of step ST101 from the storage 36, which associates and stores the input of the patient with the response in response thereto. Alternatively, the acquisition unit 34 may acquire the response information corresponding to the input information of step ST101 by using, for example, a learning model that has learned the input of the patient and the response in response thereto in advance.

In step ST104, the communication unit 35 transmits, based on the control of the controller 31, the response information acquired in step ST103 to the patient terminal 10.

In step ST105, the reception unit 32 receives the behavioral input information from the patient terminal 10 or the server 20. The behavioral input information is directed to information on the behavior performed by the patient in response to the response information transmitted in step ST104.

In step ST106, the selection unit 33 determines whether to change the chatbot based on the behavioral input information received in step ST105. The selection unit 33 may determine whether to change the chatbot based on a plurality of pieces of behavioral input information. Alternatively, the selection unit 33 may determine whether to change the chatbot based on the one or more pieces of behavioral input information received within a predetermined time period. If the chatbot is to be changed (Yes), the process proceeds to step ST107. If the chatbot is not to be changed (No), the process proceeds to step ST108.

In step ST107, the selection unit 33 changes the chatbot. In this example, the selection unit 33 may, for example, presume the preferences of the patient based on the plurality of pieces of input information and behavioral input information of the patient up to the present, and select the chatbot based on the presumed preferences of the patient. The selection unit 33 may select the chatbot based on the response information transmitted in step ST104 and the behavioral input information received in step ST105, for example, by using a learning model in which the response information, the input information, and the behavioral input information are learned in advance.

In step ST108, the controller 31 determines whether to terminate the chat. The controller 31 determines whether the end of the chat has been operated by the medical professional, for example, when the patient has been fully cured. If the chat is not terminated (No), the process returns to step ST101. If the chat is to be terminated (Yes), the process ends.

The chatbot to be selected in step ST102 and the chatbot to be changed in step ST107 may be as follows.

Five chatbots may be prepared, as an example. In this example, the frequency of appearance of the chatbots to be selected or changed may be modified.

The plurality of chatbots may extract the preferences (e.g., personality) of the patient by interacting with the patient and may be modified based on the preferences of the patient. In this example, the chatbot, in its interaction with the patient, may ask the patient, as an example, “Would you like me to explain in more detail?” and if the patient declines, the chatbot may determine that the patient does not wish to know more about the disease.

Each of the plurality of chatbots provides the patient with different patterns of contents of responses (response information) for the same content.

The plurality of chatbots communicate the contents of responses (response information) to the patient at different time points depending on the patterns. For example, in the initial stage, the time point of communicating the contents of the response (response information) to the patient is optional (random), and feedback is provided based on the input information and behavioral input information received by the information processing apparatus 30 in accordance with the response information to improve the time point of communicating the contents to the patient. In this feedback, the time period during which the chat application has not been launched on the patient terminal 10 may be included in the feedback evaluation.

A plurality of chatbots may be modified starting with a pattern of treatment. As an example, the chatbot may be modified starting from when the patient is taking the prescribed medication and when the patient is not taking the prescribed medication.

Next, advantages will be described.

The information processing apparatus 30 includes a reception unit 32 that receives input information based on input related to medical treatment in a patient terminal 10; a selection unit 33 that selects at least one chatbot in accordance with a patient from among a plurality of chatbots that differ in responses related to the medical treatment for the patient; an acquisition unit 34 that acquires response information for making a response related to the medical treatment to the input information received by the reception unit 32 in accordance with the characteristics of the chatbot selected by the selection unit 33; and a communication unit 35 that transmits the response information acquired by the acquisition unit 34 to the patient terminal 10.

That is, the information processing apparatus 30 selects a chatbot (mode) in accordance with the preferences of the patient and transmits the response information using the selected chatbot. The information processing apparatus 30 selects a chatbot in accordance with the preferences of the patient to increase the likelihood of accepting the response information of the chatbot by the patient, thereby increasing the continuation rate and effectiveness with respect to the medical treatment for the patient, i.e., the treatment continuation rate and treatment effectiveness of the patient. This allows the information processing apparatus 30 to increase the likelihood of improving the conditions of the patient.

This in turn allows the information processing apparatus 30 to use the chatbot to coach the patient (user).

In the information processing apparatus 30, the selection unit 33 may select at least one chatbot from among a plurality of chatbots having different characters in at least one of appearance, manner of response, and voice of response.

This allows the information processing apparatus 30 to select a chatbot in accordance with the preferences of the patient. As an example, if the patient prefers to be “encouraged by others,” the information processing apparatus 30 selects a chatbot that responds in such a manner, thereby increasing the treatment continuation rate and treatment effectiveness of the patient.

In the information processing apparatus 30, the selection unit 33 may select one of a plurality of chatbots having different time points of transmitting the response information to the patient terminal 10 and different manners of communicating the information to the patient recorded in the response information as well as different settings as the characteristics of the chatbot in accordance with the preferences of the patient that owns the patient terminal 10.

This allows the information processing apparatus 30 to select the chatbot in accordance with the preferences of the patient. As an example, when a patient prefers to receive an immediate response when the patient transmits input information to the information processing apparatus 30 using the patient terminal 10, the information processing apparatus 30 can select a chatbot that transmits the response information immediately to the input information as a time point for transmitting the response information. This allows the information processing apparatus 30 to increase the treatment continuation rate and treatment effectiveness of the patient.

In the information processing apparatus 30, the reception unit 32 may receive input information on medical treatment for the patient that owns the patient terminal 10, and the acquisition unit 34 may acquire response information as a proposal to improve a disease as a response regarding the medical treatment for the patient.

This allows the information processing apparatus 30 to provide medical advice to the patient. In addition, the information processing apparatus 30 allows a doctor to use the chat related to the medical treatment in responding to the input by the patient using a chatbot when the doctor examines the patient by the doctor checking the response information when the response information is selected.

In the information processing apparatus 30, the reception unit 32 may receive the behavioral input information on the behavior of the patient, and the selection unit 33 may change the chatbot to be selected based on the response information and the behavioral input information.

Even if one of a plurality of chatbots is selected and a response is made by the selected chatbot, the patient may feel reluctant to prefer the selected chatbot. Also, if chats are conducted multiple times between the patient and the chatbot, the patient may lose interest in interacting with the chatbot. In this configuration, the treatment of the patient may be liable to be interrupted. Even in such a configuration, the information processing apparatus 30 changes the chatbot, allowing the patient to continue to be treated by the chatbot that the patient prefers and also the interest of the patient to be maintained. This allows the information processing apparatus 30 to increase the treatment continuation rate and treatment effectiveness of the patient.

In the information processing apparatus 30, the reception unit 32 may receive, as the behavioral input information, behavior performed by the patient in response to the response information transmitted to the patient terminal 10 by the communication unit 35.

This allows the information processing apparatus 30 to determine whether the chatbot may be changed based on the behavioral input information.

That is, even when the information processing apparatus 30 transmits the response information, if the patient does not perform the behavior in response to the response information, the treatment continuation rate and treatment effect of the patient cannot be improved. The information processing apparatus 30 determines whether the patient is interested in the chatbot based on the behavioral input information, and if it is determined that the patient is not interested, the chatbot is changed. This allows the information processing apparatus 30 to increase the treatment continuation rate and treatment effectiveness of the patient.

In the information processing apparatus 30, the selection unit 33 may change the character to be selected based on the learning model that has learned in advance the response information in accordance with the characteristics of the chatbot and the behavior performed by the patient in response to the response information, and the behavioral input information received by the reception unit 32.

This allows the information processing apparatus 30 to change the chatbot so that the patient performs the behavior in response to the response information if the patient does not perform the behavior in response to the response information.

In the information processing apparatus 30, the reception unit 32 may receive the behavioral input information from an external device (such as, as an example, a server 20) that has stored the behavioral input information on the behavior of the patient.

The patient terminal 10 may transmit information on the behavior performed by the patient in response to the response information (behavioral input information) to the server 20 as well as the information processing apparatus 30. The reception unit 32 may receive the behavioral input information from the patient terminal 10 or from the server 20.

When the information processing apparatus 30 receives the behavioral input information from the server 20, the information processing apparatus 30 can, for example, collectively receive the behavioral input information of the patient stored during a predetermined time period or can receive the behavioral input information required by the information processing apparatus 30 from among a plurality of pieces of the stored behavioral input information.

The information processing method includes the steps of: receiving input information based on input related to medical treatment in a patient terminal 10; selecting at least one chatbot in accordance with a patient from among a plurality of chatbots that differ in responses related to the medical treatment for the patient; acquiring response information for making a response related to the medical treatment to the input information received in the receiving step in accordance with the characteristics of the chatbot selected in the selecting step; and transmitting the response information acquired in the acquiring step to the patient terminal 10.

That is, the information processing method selects a chatbot (form) in accordance with the preferences of the patient and transmits response information using the selected chatbot. The information processing method selects a chatbot in accordance with the preferences of the patient to increase the likelihood of accepting the response information of the chatbot by the patient, thereby increasing the continuation rate and effectiveness with respect to the medical treatment for the patient, i.e., the treatment continuation rate and treatment effectiveness of the patient. This allows the information processing method to increase the likelihood of improving the symptoms of the patient.

This in turn allows the information processing method to use a chatbot to coach a patient (user).

The information processing program causes a computer to execute the functions of: receiving input information based on input related to medical treatment in a patient terminal 10; selecting at least one chatbot in accordance with a patient from among a plurality of chatbots that differ in responses related to the medical treatment for the patient; acquiring response information for making a response related to the medical treatment to the input information received by the reception function in accordance with the characteristics of the chatbot selected by the selection function; and transmitting the response information acquired by the acquisition function to the patient terminal 10.

That is, the information processing program selects a chatbot (form) in accordance with the preferences of the patient and transmits response information using the selected chatbot. The information processing program selects a chatbot in accordance with the preferences of the patient to increase the likelihood of accepting the response information of the chatbot by the patient, thereby increasing the continuation rate and effectiveness with respect to the medical treatment for the patient, i.e., the treatment continuation rate and treatment effectiveness of the patient. This allows the information processing program to increase the likelihood of improving the symptoms of the patient.

This in turn allows the information processing program to use a chatbot to coach the patient (user).

Each portion of the information processing apparatus 30 described above may be embodied by an arithmetic processing unit of a computer, for example. That is, the reception unit 32, the selection unit 33, and the acquisition unit 34 of the information processing apparatus 30 may be embodied as a reception function, a selection function, and an acquisition function by an arithmetic processing unit of a computer, for example.

The information processing program may cause a computer to perform the functions described above. The information processing program may be recorded on a computer readable non-transitory recording medium such as an external memory or an optical disk.

As described above, each portion of the information processing apparatus 30 may be embodied by a computer arithmetic processing unit, for example. The arithmetic processing unit, for example, may be configured by an integrated circuit. Accordingly, each portion of the information processing apparatus 30 may be embodied as a circuit configuring an arithmetic processing unit, for example. That is, the reception unit 32, the selection unit 33, and the acquisition unit 34 of the information processing apparatus 30 may be embodied as a reception circuit, a selection circuit, and an acquisition circuit configuring an arithmetic processing unit of a computer, for example.

Further, the communication unit 35 and the storage 36 of the information processing apparatus 30 may be embodied as a communication function and a storage function including a function of an arithmetic processing unit, for example. Moreover, the communication unit 35 and the storage 36 of the information processing apparatus 30 may be embodied as a communication circuit and a memory circuit by being configured by, for example, an integrated circuit. Furthermore, the communication unit 35 and the storage 36 of the information processing apparatus 30 may be configured as a communication device and a storage device by being configured by a plurality of devices, for example.

What is claimed is:

1. An information processing apparatus comprising:
a reception unit that receives input information based on input related to medical treatment in a patient terminal;
a selection unit that selects at least one chatbot in accordance with a patient from among a plurality of chatbots having different responses related to the medical treatment for the patient;
an acquisition unit that acquires response information on the medical treatment to respond to the input information received by the reception unit in accordance with characteristics of the chatbot selected by the selection unit; and
a communication unit that transmits the response information acquired by the acquisition unit to the patient terminal.

2. The information processing apparatus according to claim 1, wherein the selection unit selects at least one chatbot from among a plurality of chatbots having different characters in at least one of appearance, manner of response, and voice of response.

3. The information processing apparatus according to claim 2, wherein the selection unit selects one of a plurality of chatbots having different time points of transmitting the response information to the patient terminal, different manners of communication to the patient recorded in the response information, and different settings as the characteristics of the chatbot in accordance with preferences of the patient that owns the patient terminal.

4. The information processing apparatus according to claim 1, wherein
the reception unit receives input information on medical treatment for the patient that owns the patient terminal, and
the acquisition unit acquires response information that is a proposal to improve a disease as a response regarding the medical treatment for the patient.

5. The information processing apparatus according to claim 1, wherein
the reception unit receives behavioral input information on behavior of the patient, and
the selection unit changes the chatbot to be selected based on the response information and the behavioral input information.

6. The information processing apparatus according to claim 5, wherein the reception unit receives the behavior performed by the patient in response to the response information transmitted by the communication unit to the patient terminal as the behavioral input information.

7. The information processing apparatus according to claim 6, wherein the selection unit changes a character to be selected based on a learning model that has learned in advance the response information in accordance with the characteristics of the chatbot and behavior performed by the patient in response to the response information, and behavioral input information received by the reception unit.

8. The information processing apparatus according to claim 5, wherein the reception unit receives the behavioral input information from an external device that stores the stored behavioral input information on the behavior of the patient.

9. An information processing method causing a computer to execute the steps of:
receiving input information based on input related to medical treatment in a patient terminal;
selecting at least one chatbot in accordance with a patient from among a plurality of chatbots having different responses related to the medical treatment for the patient;
acquiring response information on the medical treatment to respond to the input information received in the receiving step in accordance with characteristics of the chatbot selected in the selecting step; and
transmitting the response information acquired in the acquiring step to the patient terminal.

10. A non-transitory computer readable recording medium storing therein an information processing program causing a computer to execute the functions of:
receiving input information based on input related to medical treatment in a patient terminal;
selecting at least one chatbot in accordance with a patient from among a plurality of chatbots having different responses related to the medical treatment for the patient;
acquiring response information on the medical treatment to respond to the input information received by the reception function in accordance with characteristics of the chatbot selected by the selection function; and
transmitting the response information acquired by the acquisition function to the patient terminal.
